(19) United States
(12) Patent Application Publication
(10) Pub. No.: US 2021/0052336 A1
(43) Pub. Date: Feb. 25, 2021

(54) ROBOTICALLY CONTROLLED SURGICAL TOOL

(71) Applicant: Medrobotics Corporation, Raynham, MA (US)

(72) Inventors: Anish Mampetta, Waterbeach Cambridgeshire (GB); Richard Andrews, North Attelboro, MA (US); Jonathan E. Wilson, Mattapoisett, MA (US); Ian J. Darisse, Southborough, MA (US); Jesse Mitchell, Jamaica Plain, MA (US)

(21) Appl. No.: 16/960,161

(22) PCT Filed: Jan. 7, 2019
(86) PCT No.: PCT/US19/12482

§ 371 Date: Jul. 6, 2020

Related U.S. Application Data
(60) Provisional application No. 62/614,225, filed on Jan. 5, 2018.
(60) Provisional application No. 62/614,228, filed on Jan. 5, 2018.

(51) Int. Cl. A61B 34/30

(52) U.S. Cl. CPC A61B 34/30; A61B 2017/0034; A61B 2034/301

(57) ABSTRACT

A system for performing a medical procedure on a patient includes an articulating probe assembly and at least one tool. The articulating probe assembly comprises an inner probe comprising multiple articulating inner links, an outer probe surrounding the inner probe and comprising multiple articulating outer links, and at least two working channels that exit a distal portion of the probe assembly. The at least one tool is configured to translate through one of the at least two working channels. The at least one tool is robotically controlled.

### RELATED APPLICATIONS

This application claims the benefit of U.S. Provisional Application No. 62/613,899, filed Jan. 5, 2018, the content of which is incorporated herein by reference in its entirety.

This application claims the benefit of U.S. Provisional Application No. 62/614,223, filed Jan. 5, 2018, the content of which is incorporated herein by reference in its entirety.

This application claims the benefit of U.S. Provisional Application No. 62/614,224, filed Jan. 5, 2018, the content of which is incorporated herein by reference in its entirety.

This application claims the benefit of U.S. Provisional Application No. 62/614,228, filed Jan. 5, 2018, the content of which is incorporated herein by reference in its entirety.

This application claims the benefit of U.S. Provisional Application No. 62/614,225, filed Jan. 5, 2018, the content of which is incorporated herein by reference in its entirety.

This application claims the benefit of U.S. Provisional Application No. 62/614,240, filed Jan. 5, 2018, the content of which is incorporated herein by reference in its entirety.

This application claims the benefit of U.S. Provisional Application No. 62/614,235, filed Jan. 5, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, PCT Publication No. WO2015/102939, the content of which is incorporated herein by reference in its entirety.

This application is related to U.S. patent application Ser. No. 14/892,750, filed Nov. 20, 2015, U.S. Publication No. 2016/0256226, now U.S. Pat. No. 10,004,568 issued on Jun. 26, 2018, the content of which is incorporated herein by reference in its entirety.

This application is related to U.S. patent application Ser. No. 15/899,826, filed Feb. 20, 2018, U.S. Publication No. 2018/0250095 the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, PCT Publication No. WO2012/054829, the content of which is incorporated herein by reference in its entirety.

This application is related to U.S. patent application Ser. No. 13/880,525, filed Apr. 19, 2013, U.S. Publication No. 2014/0005683, now U.S. Pat. No. 8,992,421, issued on Mar. 31, 2015, the content of which is incorporated herein by reference in its entirety.

This application is related to U.S. patent application Ser. No. 14/587,166, filed Dec. 31, 2014, U.S. Publication No. 2015/0313449, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US2012/040414, filed Jun. 1, 2012, PCT Publication No. WO2012/167043, the content of which is incorporated herein by reference in its entirety.

This application is related to U.S. patent application Ser. No. 14/119,316, filed Nov. 21, 2013, U.S. Publication No. 2014/0094825, the content of which is incorporated herein by reference in its entirety.

This application is related to U.S. Provisional Application No. 62/504,175, filed May 10, 2017, the content of which is incorporated herein by reference in its entirety.

This application is related to PCT Application No. PCT/US2018/031774, filed May 9, 2018, PCT Publication No. WO2018/0020898, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, PCT Publication No. WO2012/078309, the content of which is incorporated herein by reference in its entirety.

This application is related to U.S. patent application Ser. No. 13/884,407, filed May 9, 2013, U.S. Publication No. 2014/0012288, now U.S. Pat. No. 9,649,163, issued on May 16, 2017, the content of which is incorporated herein by reference in its entirety.

This application is related to U.S. patent application Ser. No. 15/587,832, filed May 5, 2017, U.S. Publication No. 2018/0021095, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US2012/032279, filed Apr. 5, 2012, PCT Publication No. WO2012/138834, the content of which is incorporated herein by reference in its entirety.

This application is related to U.S. patent application Ser. No. 14/008,775, filed Sep. 30, 2013, U.S. Publication No. 2014/0046305, now U.S. Pat. No. 9,962,179, issued on May 8, 2018, the content of which is incorporated herein by reference in its entirety.

This application is related to U.S. patent application Ser. No. 14/944,665, filed Nov. 18, 2015, U.S. Publication No.: 2016/0066938, the content of which is incorporated herein by reference in its entirety.

This application is related to U.S. patent application Ser. No. 14/945,685, filed Nov. 19, 2015, U.S. Publication No. 2016/0066939, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US2012/054802, filed Sep. 12, 2012, PCT Publication No. WO2013/039999, the content of which is incorporated herein by reference in its entirety.

This application is related to U.S. patent application Ser. No. 14/343,915, filed Mar. 10, 2014, U.S. Publication No. 2014/0371764, now U.S. Pat. No. 9,757,856, issued on Sep. 12, 2017, the content of which is incorporated herein by reference in its entirety.

This application is related to U.S. patent application Ser. No. 15/064,043, filed Mar. 8, 2016, U.S. Publication No. 2016/0262840, now U.S. Pat. No. 9,572,628, issued on Feb. 21, 2017, the content of which is incorporated herein by reference in its entirety.

This application is related to U.S. patent application Ser. No. 15/684,268, filed Aug. 23, 2017, U.S. Publication No. 2017/0368681, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, PCT Publication No. WO2012/015659, the content of which is incorporated herein by reference in its entirety.

This application is related to U.S. patent application Ser. No. 13/812,324, filed Jan. 25, 2013, U.S. Publication No. 2014/0012287, now U.S. Pat. No. 9,901,410, issued on Feb. 27, 2018, the content of which is incorporated herein by reference in its entirety.

This application is related to U.S. patent application Ser. No. 15/874,189, filed Jan. 18, 2018, U.S. Publication No. 2018-0206923 the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US2012/070924, filed Dec. 20, 2012, PCT Publication No. WO2013/096610, the content of which is incorporated herein by reference in its entirety.

This application is related to U.S. patent application Ser. No. 14/364,195, filed Jun. 10, 2014, U.S. Publication No. 2014/0318299, now U.S. Pat. No. 9,364,955 issued on Jun. 14, 2016, the content of which is incorporated herein by reference in its entirety.

This application is related to U.S. patent application Ser. No. 15/180,503, filed Jun. 13, 2016, U.S. Publication No. 2017/0015007, now U.S. Pat. No. 9,821,477, issued on Nov. 21, 2017, the content of which is incorporated herein by reference in its entirety.

This application is related to U.S. patent application Ser. No. 15/786,901, filed Oct. 18. 2017, U.S. Publication No. 2018/0161992, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US2013/054326, filed Aug. 9, 2013, PCT Publication No. WO2014/026104, the content of which is incorporated herein by reference in its entirety.

This application is related to U.S. patent application Ser. No. 14/418,993, filed Feb. 2, 2015, U.S. Publication No. 2015/0282835, now U.S. Pat. No. 9,675,380 issued on Jun. 13, 2017, the content of which is incorporated herein by reference in its entirety.

This application is related to U.S. patent application Ser. No. 15/619,875, filed Jun. 12, 2017, U.S. Publication No. 2018/0021060, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US2014/010808, filed Jan. 9, 2014, PCT Publication No. WO2014/110218, the content of which is incorporated herein by reference in its entirety.

This application is related to U.S. patent application Ser. No. 14/759,020, filed Jul. 2, 2015, U.S. Publication No. 2015/0342690, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US2013/043858, filed Jun. 3, 2013, PCT Publication No. WO2013/184560, the content of which is incorporated herein by reference in its entirety.

This application is related to U.S. patent application Ser. No. 14/402,224, filed Nov. 19, 2014, U.S. Publication No. 2015/0150633, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US2013/038701, filed May 20, 2014, PCT Publication No. WO2014/189876, the content of which is incorporated herein by reference in its entirety.

This application is related to U.S. patent application Ser. No. 14/888,541, filed Nov. 2, 2015, U.S. Publication No. 2016/0074028, now U.S. Pat. No. 9,517,059, issued on Dec. 13, 2016, the content of which is incorporated herein by reference in its entirety.

This application is related to U.S. patent application Ser. No. 15/350,549, filed Nov. 14, 2016, U.S. Publication No. 2017/0119364, now U.S. Pat. No. 10,016,187, issued on Jul. 10, 2018, the content of which is incorporated herein by reference in its entirety.

This application is related to U.S. patent application Ser. No. 16/020,115, filed Jun. 27, 2018, U.S. Publication No. 2018/0368823, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US2014/036571, filed May 2, 2014, PCT Publication No. WO2014/179683, the content of which is incorporated herein by reference in its entirety.

This application is related to U.S. patent application Ser. No. 14/888,189, filed Oct. 30, 2015, U.S. Publication No. 2016/0067000, now U.S. Pat. No. 9,913,695, issued on Mar. 13, 2018, the content of which is incorporated herein by reference in its entirety.

This application is related to U.S. patent application Ser. No. 15/916,664, filed Mar. 9, 2018, U.S. Publication No. 2018/0256269, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US2014/067091, filed Nov. 24, 2014, PCT Publication No. WO2015/081008, the content of which is incorporated herein by reference in its entirety.

This application is related to U.S. patent application Ser. No. 15/038,531, filed May 23, 2016, U.S. Publication No. 2016/0287224, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US2015/034424, filed Jun. 5, 2015, PCT Publication No. WO2015/188071, the content of which is incorporated herein by reference in its entirety.

This application is related to U.S. patent application Ser. No. 15/315,868, filed Dec. 2, 2016, U.S. Publication No. 2017/0100197, the content of which is incorporated herein by reference in its entirety.

This application is related to U.S. patent application Ser. No. 16/225,156, filed Dec. 19, 2018, U.S. Publication No. 2019/______, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US2016/028374, filed Apr. 20, 2016, PCT Publication No. WO2016/172162, the content of which is incorporated herein by reference in its entirety.

This application is related to U.S. patent application Ser. No. 15/567,109, filed Oct. 17, 2017, U.S. Publication No. 2018-0228557 the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US2017/054297, filed Sep. 29, 2017, PCT Publication No. WO2018/064475, the content of which is incorporated herein by reference in its entirety.

This application is related to U.S. Provisional Application No. 62/517,433, filed Jun. 9, 2017, the content of which is incorporated herein by reference in its entirety.

This application is related to PCT Application No. PCT/US2018/036876, filed Jun. 11, 2018, PCT Publication No. WO2018/227180, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US2018/026016, filed Apr. 4, 2018, PCT Publication No. WO2018/187425 the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to PCT Application No. PCT/US2018/042449, filed Jul. 17, 2018, PCT Publication No. WO2019/______, the content of which is incorporated herein by reference in its entirety.

This application is related to U.S. Provisional Application No. 62/582,283, filed Nov. 6, 2017, the content of which is incorporated herein by reference in its entirety.

This application is related to U.S. Provisional Application No. 62/614,346, filed Jan. 5, 2018, the content of which is incorporated herein by reference in its entirety.

This application is related to PCT Application No. PCT/US2018/059338, filed Nov. 6, 2018, PCT Publication No. WO2019/______, the content of which is incorporated herein by reference in its entirety.

This application is related to U.S. Design application Ser. No. 29/632,148, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### BACKGROUND

As less invasive medical techniques and procedures become more widespread, medical professionals such as surgeons may require articulating surgical tools, such as endoscopes, to perform such less invasive medical techniques and procedures that require access to locations within the patient, such as a site accessible through the mouth or other natural orifice, or a site accessible through an incision through the patient's skin.

There is a need for improved systems for performing a medical procedure.

### SUMMARY

In an aspect, a system for performing a medical procedure on a patient comprises: an articulating probe assembly, comprising: an inner probe comprising multiple articulating inner links; an outer probe surrounding the inner probe and comprising multiple articulating outer links; at least two working channels that exit a distal portion of the probe assembly, and at least one tool configured to translate through one of the at least two working channels, wherein the at least one tool is robotically controlled.

In an embodiment, the at least one tool comprises: an interface assembly at a proximal end; a shaft extending in a distal direction from the interface assembly; an end effector at a distal end of the shaft; and an articulation region comprising a plurality of links that articulate relative to one another.

In an embodiment, the interface assembly comprises: at least one rotatable capstan; and at least one cable, the at least one cable extending from a corresponding one of the at least one capstans through the shaft and into the articulation region.

In an embodiment, the at least one cable is selectively tensioned and de-tensioned by the capstan to control an articulation of the articulation region.

In an embodiment, the at least one cable comprises a first set of cables and a second set of cables that are selectively tensioned and de-tensioned by a corresponding set of first capstans and second capstans respectively to control an articulation of a first articulation portion and to control an articulation of a second articulation portion of the articulation region.

In an embodiment, the at least one cable is selectively tensioned and de-tensioned by the capstan to control an operation of the end effector.

In an embodiment, the at least one cable is electrically conductive to deliver electromagnetic energy from the interface assembly to the end effector.

In an embodiment, the interface assembly comprises a rotating assembly that rotates about a center axis of the tool.

In an embodiment, the at least one capstan is positioned on the rotating assembly.

In an embodiment, the rotating assembly comprises an outer rotating assembly and an inner rotating assembly, and wherein the outer rotating assembly and inner rotating assembly independently rotate about the center axis of the tool.

In an embodiment, the at least one capstan comprises a first capstan positioned on the outer rotating assembly and a second capstan positioned on the outer rotating assembly.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a schematic view of a system in which embodiments of the present inventive concepts can be practiced.

FIGS. 1A-C are graphic demonstrations of a robotic probe, in accordance with embodiments of the present inventive concepts.

FIG. 2A is a perspective view of a tool, in accordance with embodiments of the present inventive concepts.

FIG. 2B is a perspective cross-sectional view of a proximal portion of the tool of FIG. 2A, in accordance with embodiments of the present inventive concepts.

FIG. 3A is a perspective of view of an inner rotating assembly, in accordance with embodiments of the present inventive concepts.

FIGS. 3B and 3C are front and back perspective views respectively of the hub of the inner rotating assembly in accordance with embodiments of the present inventive concepts.

FIG. 3D is a cross-sectional view of the hub, in accordance with embodiments of the present inventive concepts.

FIGS. 3E and 3F are a perspective view and a perspective cross-sectional view of the hub, in accordance with embodiments of the present inventive concepts.

FIG. 4 is a perspective view of a capstan, in accordance with embodiments of the present inventive concepts.

FIG. 5 is a perspective cross-sectional view of a radial adapter, in accordance with embodiments of the present inventive concepts.

FIG. 6 is a perspective partial cut away view of a portion of a shaft of the tool, in accordance with embodiments of the present inventive concepts.

FIGS. 7A-C are a rear perspective view, a cutaway rear perspective view, and a front perspective view of an outer rotating assembly, in accordance with embodiments of the present inventive concepts.

FIG. 8 is a perspective view of a second hub of the outer rotating assembly, in accordance with embodiments of the present inventive concepts.

FIG. 9 is a partially transparent perspective view of a radial adapter, in accordance with embodiments of the present inventive concepts.

FIG. 10 is a perspective view of the distal portion of the tool including the articulating portion and the end effector, in accordance with embodiments of the present inventive concepts.

FIG. 10A is a perspective, partial sectional view of the multiple articulatable links in accordance with embodiments of the present inventive concepts.

FIG. 10B is a close-up perspective view of two neighboring links, in accordance with embodiments of the present inventive concepts.

FIGS. 11A-D are perspective, side-sectional, top-sectional and partial-sectional views of a distal portion of the tool, and related jaw and control cables, in accordance with embodiments of the present inventive concepts.

### DETAILED DESCRIPTION OF EMBODIMENTS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate” shall include locations relatively close to, on, in and/or within a referenced component, anatomical location, or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

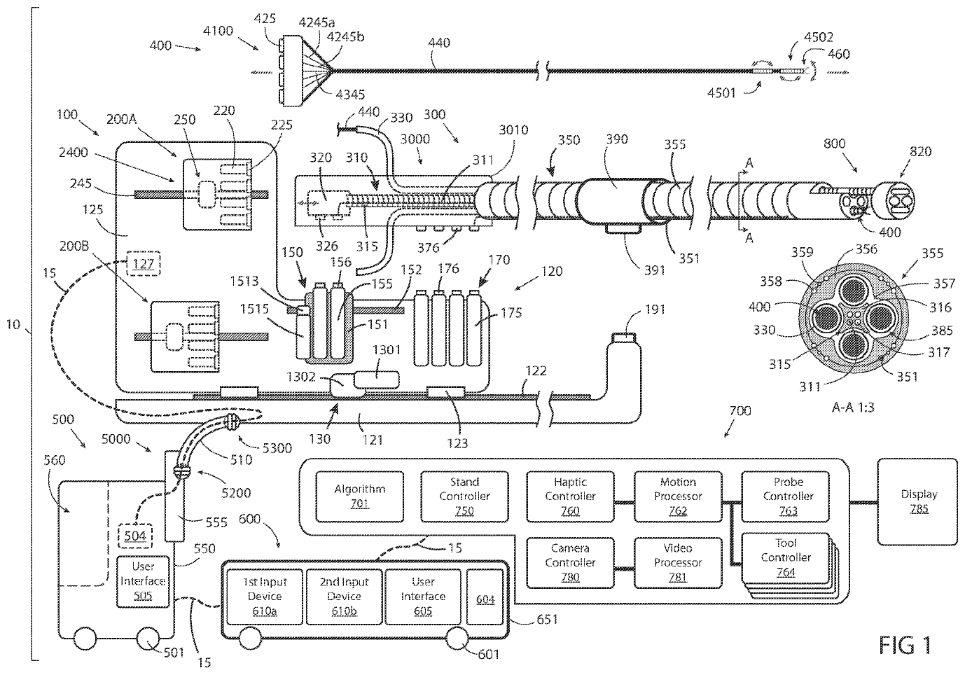

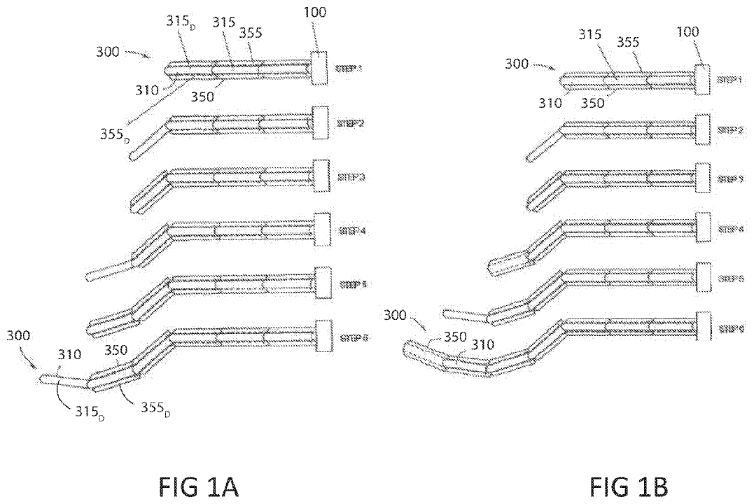

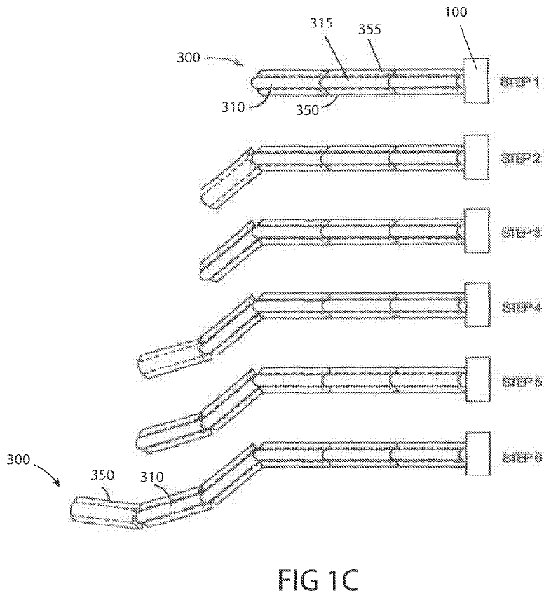

Referring to FIG. 1, a schematic view of a system in which embodiments of the present inventive concepts can be practiced is illustrated.

System 10 includes a robotic feeder 100. Feeder 100 interchangeably and operably engages a robotic probe assembly 300, and at least one robotic tool assembly 400. Feeder 100 is constructed and arranged to advance, retract, steer, and/or otherwise control the position and/or articulation of probe assembly 300 and/or tools 400, as described herein. One or more tools 400 can be slidingly received within a channel of probe assembly 300, and each tool 400 can be advanced beyond the distal end of probe assembly 300. Feeder 100 includes a probe manipulation assembly 120 for operably controlling the position and articulation of probe assembly 300. Feeder 100 also includes at least one tool manipulation assembly, tool drive 200 (e.g. tool drives 200A and 200B shown), for controlling the position and articulation of an attached tool 400. System 10 further includes a multi-dimensional positioning assembly, stand 500. Stand 500 includes an articulation assembly 5000 for positioning feeder 100 with multiple degrees of freedom, for example within an operating room, relative to a patient and/or patient bed, as described herein. System 10 further includes a control interface, surgeon console 600, configured to receive commands from one or more operators of system 10 (e.g. one or more surgeons or other clinicians). Console 600 can include a first and second input device, 610A and 610B respectively (singly or collectively input devices 610 herein), each configured to receive multi-dimensional input data (e.g. via a kinematic input device as described herein). System 10 further includes a collection of data processing components, collectively processing unit 700. Processing unit 700 can include one or more algorithms, controllers, memory, state machines, and/or processors, singly and/or collectively controlling one or more components of system 10 (e.g. based at least on one or more user inputs received by one or more input components of system 10). System 10 further includes an imaging device, camera assembly 800 (e.g. a tool 400 configured as a camera, as described herein), comprising one or more cameras, camera 820. Image data (e.g. still and/or video images) captured by camera 820 can be displayed on one or more monitors or other screens, display 785. One or more components described herein as included in a tool 400 can also be included in camera assembly 800, for example camera assembly 800 can comprise a tool 400 with camera 820 operably attached thereto. A conduit, bus 15, can connect one or more components of system 10. Bus 15 can comprise one or more electrical, fluid, optical, and/or other conduits for transferring information, power, one or more fluids, light energy, and combinations of one or more of these.

### Probe Assembly 300

Probe assembly 300 includes an outer probe 350, comprising multiple articulating outer links 355. Links 355 each comprise a ring-like structure (e.g. a hollow tube-like structure), link body 356, surrounding a hollow bore, channel 357. Collectively, channels 357 define a lumen extending along at least a portion of the length of outer probe 350. Links 355 can include multiple lumens extending therethrough, such as lumens extending along the link, through link body 356. For example, links 355 can include one or more steering cable lumens, lumens 358, such as eight lumens 358 shown. Lumens 358 can each slidingly receive a steering cable 351 that is used to control at least the articulation of outer probe 350, as described herein. Links 355 can also include one or more auxiliary lumens, four lumens 359 shown. In some embodiments, lumens 359 can slidingly receive elongate devices and/or filaments, such as optical fibers for delivering light to a surgical site.

Probe assembly 300 further includes inner probe 310, comprising multiple articulating inner links 315. Inner probe 310 is slidingly received within channels 356 extending through outer probe 350. Links 315 can comprise a link body 316, and can include multiple lumens extending therethrough, such as lumens extending along the link. For example, links 315 can include one or more steering cable lumens, lumens 317, such as four lumens 318 shown. Lumens 317 can each slidingly receive a steering cable 311 used to control at least the articulation of inner probe 310, as described herein.

The outer shape of link body 316 can align with the shape of the channel 357 to form a plurality of passageways or working channels 385, extending throughout probe assembly 300. Working conduits 330 can be slidingly received within channels 385, extending throughout the probe assembly 300. Each conduit 330 can sliding receive at least a portion of a tool 400.

Probe assembly 300 can be of similar construction and arrangement to the similar device described in reference to applicant's co-pending U.S. patent application Ser. No. 16/114,681, filed Aug. 28, 2018, the content of which is incorporated herein by reference in its entirety.

Probe assembly 300 further comprises a manipulation assembly 3000, operably attached to the proximal portion of probes 310, 350. Manipulation assembly 3000 comprises a housing 3010, surrounding at least a cart 320, operably attached to inner probe 310. Manipulation assembly 3000 comprises one or more bobbins 376 operably attached to one or more steering cables 351 (also referred to herein as control cables). Cart 320 comprises one or more bobbins 326 operably attached to one or more steering cables 311. Manipulation assembly 3000 is constructed and arranged to operably and removably attach to feeder 100, as described herein. Manipulation assembly 3000 supports the proximal sections of one or more working conduits 330 in an orientation that is radially dispersed relative to the radially compact orientation of the distal portions of working conduits 330 within probe assembly 300.

Probe assembly 300 can include a support structure, introducer 390. Introducer 390 can comprise a rigid elongate structure. Introducer 390 can surround at least a portion of probe assembly 300. Introducer 390 can comprise a connector portion 391, constructed and arranged to operably attach to a portion of feeder 100 as described herebelow.

Probe assembly 300 can be of similar construction and arrangement to the similar device described in applicant's co-pending application U.S. Provisional Application No. 62/614,240, filed Jan. 5, 2018, the content of which is incorporated herein by reference in its entirety.

### Feeder 100

Feeder 100 comprises a manipulation assembly 120 comprising a carriage 125 operably attached to a base 121. Carriage 125 can comprise one or more linear bearings 123 fixedly attached thereto, slidingly attached to a linear rail assembly 122, which in turn is fixedly attached to base 121. Linear rail assembly 122 can comprise one or more rails and/or lead screws. Manipulation assembly 120 can comprise a linear drive assembly 130, that is operably attached to carriage 125 and linear rail assembly 122. For example, linear rail assembly 122 can comprise at least a lead screw, and linear drive assembly 130 can comprise a motor 1301 and gear box 1302. Linear drive assembly 130 can be configured to engage the lead screw of linear rail assembly 122, such as to translate carriage 125 relative to base 121.

Manipulation assembly 120 can comprise a probe support assembly 170. Probe support assembly 170 can comprise at least a portion of carriage 125. Probe support assembly 170 can comprise one or more motors 175, each operably attached to a capstan 176. Probe support assembly 170 is constructed and arranged to operably and removably attach to manipulation assembly 3000, for example, such that each capstan 176 operably engages a corresponding bobbin 376. Motors 175 can be configured to rotate capstans 176, which in turn rotate bobbins 376, tensioning and de-tensioning cables 351 to control the articulation of outer probe 350.

Probe support assembly 170 can further comprise a probe translation assembly 150. Probe translation assembly 150 can comprise one or more motors 155, each operably attached to a capstan 156. Probe translation assembly 150 is constructed and arranged to operably and removably attach to cart 320, for example such that each capstan 156 operably engages a corresponding bobbin 326. Motors 155 can be configured to rotate capstans 156, which in turn rotate bobbins 326, tensioning and de-tensioning cables 311 to control the articulation of inner probe 310. Probe translation assembly 150 can comprise a cart 151. Motors 155 can be fixedly attached to cart 151. Cart 151 can be slidingly attached to a linear rail assembly 152, fixedly attached to carriage 125. Linear rail assembly 152 can comprise one or more rails and/or lead screws. Probe translation assembly 150 can comprise a motor 1515 and drive gear 1513 operably attached thereto. Drive gear 1513 can operably attach to linear rail assembly 152, for example when linear rail assembly 152 comprises at least a lead screw. Motor 1515 can be configured to rotate drive gear 1513 to translate cart 151 relative to carriage 125. Cart 151 can be constructed and arranged to engage cart 320, such that translation of cart 151 causes the translation of cart 320 within manipulation assembly 3000. Translation of cart 320 can cause the translation of inner probe 310 with respect to outer probe 350, as described herein.

Feeder 100 can include a connector portion 191, constructed and arranged to removably connect to introducer 390 of probe assembly 300. Connector portion 191 can be positioned at the distal end of carriage 125, as shown.

Feeder 100 can include one or more modules 127, such as one or more processors and/or controllers. Module 127 can be operably attached to one or more components of system 10 via bus 15.

Feeder 100 can be of similar construction and arrangement to the similar device described in applicant's co-pending application U.S. Provisional Application No. 62/614,240, filed Jan. 5, 2018, the content of which is incorporated herein by reference in its entirety.

### Tool Drive 200

Each tool drive 200 (also referred to herein as a singular tool drive 200) is configured to operably and interchangeably attach to one or more tools 400. Feeder 100 can comprise one, two, three, four, or more tool drives, tool drives 200A and 200B shown. Additional tool drives can be mounted to carriage 125 opposite tool drives 200A and 200B (e.g. on the opposite side of carriage 125). Tool drive 200 can slidingly attach to carriage 125 via a translation assembly 2400. Translation assembly 2400 can comprise a linear rail assembly 245, fixedly attached to carriage 125. Linear rail assembly 245 can comprise one or more rails and/or lead screws. Translation assembly 2400 can further comprise a linear drive assembly 250, operably attached to tool drive 200 and linear rail assembly 245. For example, linear rail assembly 245 can comprise at least a lead screw, and linear drive assembly 250 can comprise a motor and/or a gear box. Linear drive assembly 250 can be configured to engage the lead screw of linear rail assembly 245, to translate tool drive 200 relative to carriage 125. Translation of tool drive 200 can cause the translation of an attached tool 400, for example relative to outer probe 350 operably attached to manipulation assembly 120.

Tool drive 200 can comprise one or more motors 220, configured to manipulate one or more components of tool drive 200. For example, one or more motors 220 can be configured to rotate one or more assemblies of tool drive 200 relative to each other, and/or to rotate one or more gears 225 (e.g. capstans) of tool drive 200. Gears 225 of tool drive 200 can be configured to operably engage one or more bobbins of an attached tool 400, as described herein, to control the articulation of the attached tool 400.

Tool drive 200 can be of similar construction and arrangement to the similar device described in applicant's co-pending application U.S. Provisional Application No. 62/614,228, filed Jan. 5, 2018, the content of which is incorporated herein by reference in its entirety.

### Tool 400

Tool 400 can include a manipulation assembly 4100, operably attached to the proximal end of a shaft 440. Shaft 440 can comprise a flexible shaft, comprising one or more lumens. Tool 400 can comprise one or more sets of steering (or control) cables 4245a, 4245b, and or 4345. Cables 4245a,b can be operably attached to manipulation assembly 4100, and extend through shaft 440 to a first and second articulation section 4501 and 4502, respectively. Cables 4245a,b can be tensioned and/or de-tensioned by manipulation assembly 4100 to cause the articulation of articulation sections 4501 and 4502, respectively. Cables 4345 can be operably attached to manipulation assembly 4100, and extend through shaft 440 to an end effector 460. Cables 4345 can be tensioned and/or de-tensioned by manipulation assembly 4100 to cause the articulation or other manipulation of end effector 460. System 10 can comprise multiple tools 400, such as four, five, six, or more tools 400, each exchangeable and operably attachable to tool drives 200. End effectors 460 can comprise scissors, graspers, blades, cautery devices, laser devices, and the like. Manipulation assembly 4100 can be constructed and arranged to removably attach to tool drive 200, such that gears 225 engage bobbins 425 of manipulation assembly 4100. Motors 220 of tool drive 200 can rotate gears 225, and bobbins 425, to tension and/or de-tension one or more cables of tool 400 described herein, to tension and/or de-tension the cables and manipulate tool 400. Manipulation assembly 4100 can also be constructed and arranged to rotate one or more components of tool 400 relative to each other, for example to rotate end effector 460 relative to shaft 440.

Tool 400 can be of similar construction and arrangement to the similar device described in applicant's co-pending application U.S. Provisional Application No. 62/614,225, filed Jan. 5, 2018, the content of which is incorporated herein by reference in its entirety.

### Camera Assembly 800

In some embodiments, as described hereabove, a tool 400 can be configured as a camera assembly 800. Camera assembly 800 can comprise a camera 820, operably attached to the distal end of shaft 440 of a tool 400. In some embodiments, camera 820 is attached to shaft 440 after shaft 440 has been inserted through probe assembly 300. For example, in some embodiments, camera 820 is larger than working channel 385.

Camera assembly 800 can be of similar construction and arrangement to the similar device described in applicant's co-pending application PCT International Patent Application No. PCT/US2018/059338, filed Nov. 6, 2018, the content of which is incorporated herein by reference in its entirety.

### Stand 500

Stand 500 can be constructed and arranged to position feeder 100 relative to a patient and/or patient bed, such as to position probe assembly 300 for a surgical procedure. For example, surgical procedures can include but are not limited to transabdominal procedures, transoral procedures, trans anal procedures, and/or trans vaginal procedures. Stand 500 includes a base 550, supporting an articulation assembly 5000. Articulation assembly 5000 includes a tower 555, extending vertically from base 550. A first hub 5200 is operably attached to tower 555. First hub 5200 can be adjusted along the height of tower 555, via one or more motors and/or vertical translation assemblies. First hub 5200 is operably attached to positioning arm 510, which is operably attached to a second hub 5300. Second hub 5300 is operably attached to base 121 of feeder 100. Hubs 5200 and 5300 can each comprise one or more motors, gears, hinges, axles, and the like, configured to manipulate the position of feeder 100 relative to stand 500. Bus 15 of system 10 can operably connect feeder 100 to stand 500. In some embodiments, bus 15 is routed through hubs 5200, 5300, arm 510, and/or tower 555, such that bus 15 is at least partially contained within articulation assembly 5000.

Stand 500 can comprise a recess 560. Articulation assembly 5000 can be configured to “fold” into a stowed position, with feeder 100 positioned at least partially within recess 560. In some embodiments stand 500 can comprise a processor 504 and a user interface 505. User interface 505 can include input and output functionality, such as a touchscreen monitor. User interface 505 can be configured to allow a user to control one or more components of system 10, for example the articulation of articulation assembly 5000. In some embodiments, stand 500 includes one or more wheels 501, and is constructed and arranged to be mobile. For example, stand 500 can be manually repositionable by a user and/or can be robotically repositionable, for example when wheels 501 are driven by one or more motors.

Stand 500 can be of similar construction and arrangement to the similar device described in applicant's co-pending application U.S. Provisional Application No. 62/614,223, filed Jan. 5, 2018, the content of which is incorporated herein by reference in its entirety.

### Surgeon Console 600

Surgeon console 600 can be operably attached to one or more components of system 10, such as via bus 15. Console 600 can comprise a base 651, supporting input devices 610a,b, and user interface 605. Console 600 can comprise a processor 604. In some embodiments, processor 604 can receive commands from input device 610a,b, and/or user interface 605. User interface 605 can be configured to allow a user to control one or more components of system 10. In some embodiments, user interface 605 can be a redundant interface of user interface 505, such that a user can perform the same operations from either interface. In some embodiments, console 600 includes one or more wheels 601, and is constructed and arranged to be mobile. For example, console 600 can be manually repositionable by a user and/or can be robotically repositionable, for example when wheels 601 are driven by one or more motors.

Console 600 can be of similar construction and arrangement to the similar device described in applicant's co-pending application U.S. Provisional Application No. 62/614,224, filed Jan. 5, 2018, the content of which is incorporated herein by reference in its entirety.

### Processor 700

Processing unit 700 can comprise one or more controllers and/or processors, located throughout system 10. For example, processor 700 can comprise a computer or other processing device, and/or can comprise one or more controllers or modules of system 10 (e.g. module 127 of feeder 100, processor 504 of stand 500, and/or processor 604 of user interface 600). Processing unit 700 can comprise one or more algorithms for processing data and/or commanding one or more components of system 10 to perform one or more operations. Processing unit 700 can comprise one or more controllers for controlling components of system 10. Processing unit 700 can comprise a stand controller 750, for operational control of stand 500. Processing unit 700 can comprise a camera controller, for operational control of camera assembly 800. Camera controller 780 can be operably attached to a video processor 781 for processing image data captured by camera 820. Video processor 781 can provide processed image data to a display 785, for display to a user. Processing unit 700 can comprise a haptic controller 760, operably attached to input devices 610a,b of console 600, for example via processor 604. Haptic controller 760 can be operably attached to a motion processor 762, which is operably attached to a probe controller 763, and one or more tool controllers 764. Haptic controller 760 can receive multi-dimensional input data (e.g. via a kinematic input device) from input devices 610a,b, and/or provide haptic feedback commands to input devices 610a,b. Motion processor 762 can process the multi-dimensional input data, and provide articulation and/or translation commands to probe controller 763 and/or tool controllers 764. Probe controller 763 can provide commands to one or more motors of system 10, for example to one or more motors of manipulation assembly 120 to at least advance, retract, steer, and/or otherwise control the position and/or articulation of probe assembly 300. Tool controllers 764 can provide commands to one or more motors of system 10, for example one or more motors of a tool drive 200 to at least advance, retract, steer, and/or otherwise control the position and/or articulation of an attached tool 400.

Processor 700 can be of similar construction and arrangement to the similar device described in applicant's co-pending application U.S. Provisional Application No. 62/614,235, filed Jan. 5, 2018, the content of which is incorporated herein by reference in its entirety.

Referring additionally to FIGS. 1A-C, graphic demonstrations of a robotic probe 300 are illustrated, consistent with the present inventive concepts. Articulating probe 300 comprises essentially two concentric mechanisms, an outer mechanism and an inner mechanism, each of which can be viewed as a steerable mechanism. Each of the components of probe 300 can comprise one or more sealing elements, such as to support an insufflation procedure. FIGS. 1A-C show the concept of how different embodiments of robotic probe 300 operate. Referring to FIG. 1A, the inner mechanism can be referred to as a first mechanism or inner probe 310. The outer mechanism can be referred to as a second mechanism or outer probe 350. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume outer probe 350 is rigid and inner probe 310 is limp, as seen in step 1 in FIG. 1A. Now, inner probe 310 is both pushed forward by feeder 100, and a distal-most inner link 315D is steered, as seen in step 2 in FIG. 1A. Now, inner probe 310 is made rigid and outer probe 350 is made limp. Outer probe 350 is then pushed forward until a distal-most outer link 355D catches up to the distal-most inner link 315D (e.g. outer probe 350 is coextensive with inner probe 310), as seen in step 3 in FIG. 1A. Now, outer probe 350 is made rigid, inner probe 310 limp, and the procedure then repeats. One variation of this approach is to have outer probe 350 be steerable as well. The operation of such a device is illustrated in FIG. 1B. In FIG. 1B it is seen that each mechanism is capable of catching up to the other and then advancing one link beyond. According to one embodiment, outer probe 350 is steerable and inner probe 310 is not. The operation of such a device is shown in FIG. 1C.

In medical applications, operation, procedures, and so on, once robotic probe 300 arrives at a desired location, the operator, such as a surgeon, can slide one or more tools through one or more working channels of outer probe 350, inner probe 310, or one or more working channels formed between outer probe 350 and inner probe 310, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of robotic probe 300, such as working channels comprising one or more radial projections extending from outer probe 350, these projections including one or more holes sized to slidingly receive one or more tools. As described with reference to other embodiments, working channels may be positioned on other locations extending from, on, in, and/or within robotic probe 300.

Inner probe 310 and/or outer probe 350 are steerable and inner probe 310 and outer probe 350 can each be made both rigid and limp, allowing robotic probe 300 to drive anywhere in three-dimensions while being self-supporting. Articulating probe 300 can “remember” each of its previous configurations and for this reason, robotic probe 300 can retract from and/or retrace to anywhere in a three-dimensional volume such as the intracavity spaces in the body of a patient such as a human patient.

Inner probe 310 and outer probe 350 each include a series of links, i.e. inner links 315 and outer links 355 respectively, that articulate relative to each other. In some embodiments, outer links 355 are used to steer and lock robotic probe 300, while inner links 315 are used to lock robotic probe 300. In a “follow the leader” fashion, while inner links 315 are locked, outer links 355 are advanced beyond the distal-most inner link 315D. Outer links 355 are steered into position by the system steering cables, and then locked by locking the steering cables. The cable of inner links 315 is then released and inner links 315 are advanced to follow outer links 355. The procedure progresses in this manner until a desired position and orientation are achieved. The combined inner links 315 and outer links 355 may include working channels for temporary or permanent insertion of tools at the surgery site. In some embodiments, the tools can advance with the links during positioning of robotic probe 300. In some embodiments, the tools can be inserted through the links following positioning of robotic probe 300.

One or more outer links 355 can be advanced beyond the distal-most inner link 315D prior to the initiation of an operator controlled steering maneuver, such that the quantity extending beyond the distal-most inner link 315D will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of robotic probe 300. In some embodiments, an operator can select the number of links used for steering (e.g. to select between 1 and 10 links to be advanced prior to each steering maneuver).

In some embodiments, instruments are controlled through a series of cables. For example, ten (10) 0.016″ diameter 7×7 wound steel cables may be employed and tensioned by eight (8) outer capstans and four (4) inner capstans. In some embodiments, the eight (8) outer articulation cables are terminated in the articulation sections of the instrument with a 0.040″ diameter swaged steel ball that engages with bi-directional flex links and creates a pivot point for each joint. In some embodiments, the links at which the articulation cables are terminated define each joint. The two (2) inner jaw cables can be twice as long as the outer cables, and are swaged at their centers with a 0.040″ diameter steel ball that engages in a mating feature in the end effector. Each end of the inner cables is tensioned by one of the (4) inner capstans. This is referred to as a “clothesline”-type end effector design.

In some embodiments, the eight (8) articulation cables travel through flex links and into the “outer cable management”—an extruded tube consisting of eight (8) individual cable lumen dispersed radially about a larger center lumen. The cable management lumen separate the articulation cables and prevent them from crossing or twisting. At the proximal end of the outer cable management, the wires are fed through a “nose cone” which separates them further and routes them towards their respective capstans.

In some embodiments, the two (2) inner jaw cables travel through their own inner cable management, an extruded tube consisting of four (4) individual cable lumen. The inner cable management is housed inside of the inner rotation torque tube and separates the jaw cables from crossing or twisting. The inner cable management also operates as a dielectric, preventing jaw cables in bi-polar instruments from shorting together. At the proximal end of the inner cable management the wires are fed through a “diffuser” which separates them further and routes them towards their respective capstans.

In some embodiments, instruments are equipped with outer rotation. Beginning at the instrument drive, torque is transmitted through the proximal assembly and outer shaft of the instrument to rotate along its entire length. The outer shaft of the instrument can comprise a triple wire wound hollow tube with the characteristics of flexibility and good torque transmission.

In some embodiments, instruments are equipped with inner rotation. Beginning at the instrument drive, torque is transmitted through the proximal assembly and inner rotation torque tube of the instrument to rotate only the clevis and end effectors at the distal tip of the instrument—without rotating the articulation sections.

In some embodiments, the inner rotation torque tube is a hybrid construction of two triple wire wound hollow shafts with differing stiffness. The proximal section (˜1000 mm long) transmits torque well with minimal windup, but is somewhat stiffer than the distal section. The distal section (˜125 mm long) transmits torque but has increased windup, and is considerably more flexible than the proximal section. The two sections are laser welded together. The proximal section extends from the proximal assembly and stops at the distal end of the outer shaft. The distal section extends through the flex joints and terminates at the end effector clevis. This construction maximizes torque transmission, minimizes windup and allows the articulation section greater flexibility.

In some embodiments, instruments are equipped with a locking feature that prohibits the rotation of the inner with respect to the outer. This is used to key the instrument during installation and removal from the instrument drive—ensuring that the motors are properly aligned to the correct capstans. Once the instrument is installed onto the drive, the user can toggle a thumb slider that disengages the lock and allows the inner to rotate freely.

In some embodiments, the instruments are equipped with a “super outer” ring that rotates independent of the inner and outer rings. In this embodiment, this “super outer” ring can be used solely to lock the instrument to the instrument drive.

In some embodiments, instruments have three “joints”. The proximal (shoulder) joint consists of 10 bidirectional flex links. The distal (elbow) joint consists of 15 bidirectional flex links. The third joint (wrist) is considered to be the motion of the jaws about their pivot axis (clevis pin).

In some embodiments, jaws are independently controlled through a “clothesline” mechanism. This enables each jaw to open and close independent of the other, and enables “wrist” movement of the end effector.

In some embodiments, inside the distal link of the instruments there is a custom designed thrust bearing. This prevents the clevis from “sticking” to the distal link due to increased friction during cable tensioning. This enables inner rotation while the jaws are tensioned in the “open” or “closed” positions.

In some embodiments, instruments are equipped with both monopolar and bipolar cautery capability. Jaw cables can be energized to enable cautery. The bipolar energy path can be as follows: cautery cable is plugged into connector outside of instrument skin. Cable brings energy through instrument skin and to a slip ring mounted within the proximal assembly; energy is transferred through the slip ring to a copper “brush” that makes contact with an elongated screw threaded through a jaw capstan; the screw captures the jaw cable and energy travels through the cable up to the end effectors; each jaw cable is independently energized in the bipolar design; the jaws are isolated from each other by a plastic washer; the monopolar energy path in the scissor instrument differs from the bipolar instruments in that only one of the jaw cables is energized and the blades are not isolated from each other; the monopolar energy path in the hook instrument differs from the jawed instruments in that is uses an insulated wire to bring energy from the capstan screw to the end effector in place of jaw cables; the slip ring allows for infinite inner rotation of the end effector without the need for a service loop; the copper “brush” allows for infinite rotation of the cable capstan without the need for a service loop; and in the future, ideally the instrument would be energized through the instrument drive and not require an external plug.

In some embodiments, the inner rotation torque tube, slip ring and “spline” are bonded together inside the proximal assembly and attached to a sliding cross-member. This design allows for torque transmission as well as linear translation of the bonded components. The torque transmission is required for inner rotation of the end effector. Linear translation is required to compensate for compression in the outer shaft when the instrument is tensioned and when it navigates through a tortuous path. The sliding action of this sub-assembly prevents the inner rotation torque tube from bottoming out against the proximal assembly as the outer shaft compresses.

In some embodiments, instrument capstans are equipped with a rubber O-ring around their diameter to restrict them from unwinding while instrument is on the table. The O-rings provide enough friction to prevent the capstans from unwinding themselves but not so much that the motors cannot overcome them.

In some embodiments, the capstans are provided with a flange to prevent the instrument cables from slipping down the shaft of the capstan and becoming jammed in the bearings.

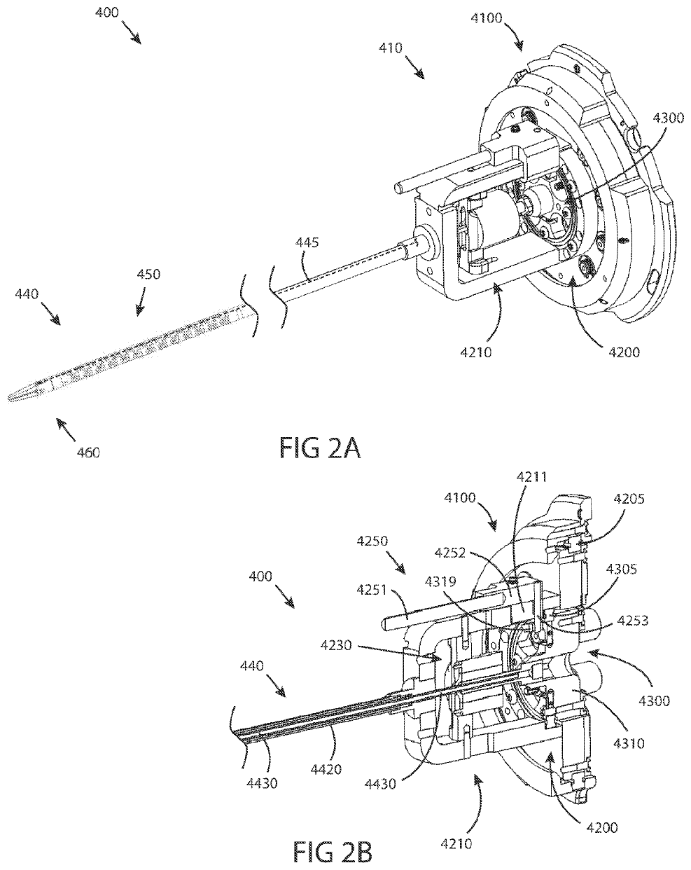

Referring to FIG. 2A, a perspective view of a tool 400 is illustrated, in accordance with embodiments of the present inventive concepts. In some embodiments, the tool 400 comprises a control assembly 410. In some embodiments, the control assembly 410 comprises an interface assembly 4100, an outer rotating assembly 4200, and an inner rotating assembly 4300.

In some embodiments, the outer rotating assembly 4200 may be rotatably positioned within the interface assembly 4100. In some embodiments, the inner rotating assembly 4300 may be rotatably positioned within the outer rotating assembly 4200. In some embodiments, the outer rotating assembly 4200 surrounds the inner rotating assembly 4300.

In some embodiments, the outer rotating assembly 4200 further comprises a support assembly 4210. The support assembly 4210 may extend from the outer rotating assembly 4200. In some embodiments, the support assembly 4210 is operably attached to a shaft 440.

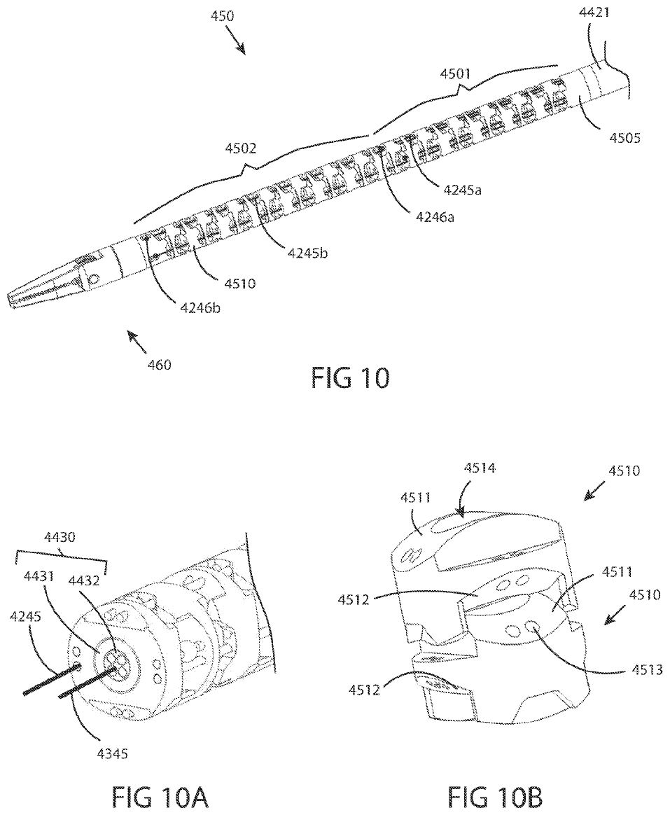

In some embodiments, the shaft 440 extends from the control assembly 410 at a proximal end of the shaft 440 to an articulating section 450 at a distal end of the shaft 440. The articulating section 450 may comprise multiple articulatable links 4510, as described herebelow in reference to FIG. 10. In some embodiments, the multiple articulatable links 4510 may be operably attached to an end effector 460.

Referring additionally to FIG. 2B, a perspective cross-sectional view of a proximal portion of a tool 400 is illustrated, in accordance with embodiments of the present inventive concepts. In the embodiment shown, a first bearing 4205 is positioned between the interface assembly 4100 and the outer rotating assembly 4200. In some embodiments, a second bearing 4305 is positioned between the outer rotating assembly 4200 and the inner rotating assembly 4300. In some embodiments, the interface assembly 4100, the outer rotating assembly 4200, and the inner rotating assembly 4300 are free to rotate relative to each other.

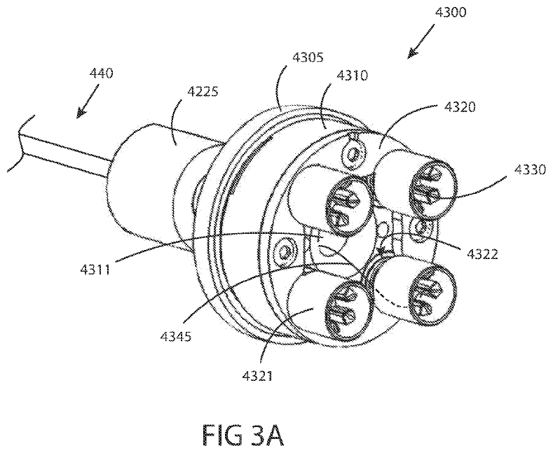

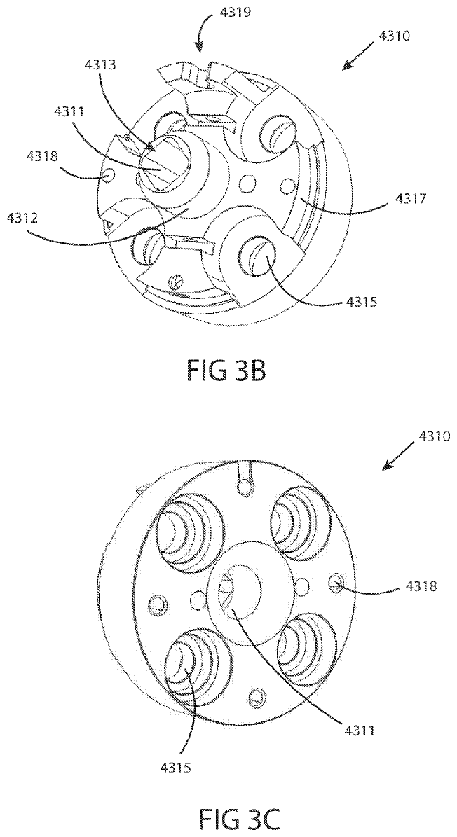

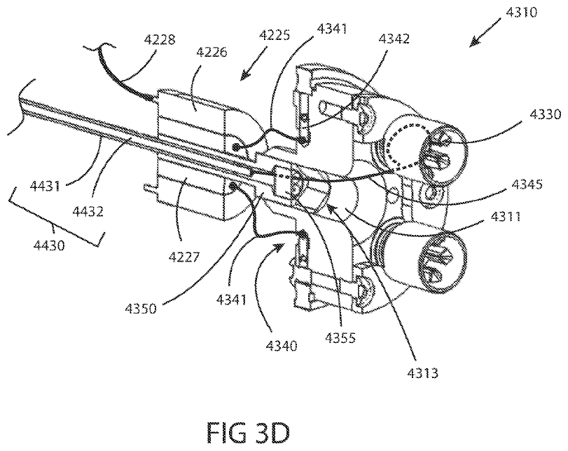

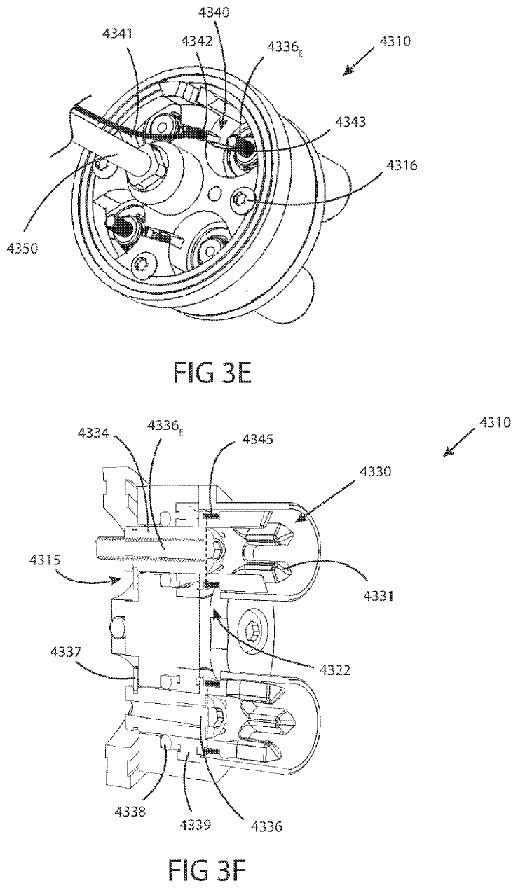

In some embodiments, the inner rotating assembly 4300 is constructed and arranged to operably engage (e.g. control the tension of) one or more control cables 445 of the tool 400, such as four cables 4345 as described herebelow in reference to FIG. 3A, the cables 4345 in turn being operably attached to an end effector 460 of the tool 400. In some embodiments, the inner rotating assembly 4300 is constructed and arranged to rotate, relative to the outer rotating assembly 4200, while maintaining the operative engagement of one or more cables 4345. In some embodiments, the rotation of the inner rotating assembly 4300 also rotates an inner shaft assembly 4430 and the end effector 460. The components of the inner rotating assembly 4300 are described in detail herebelow in reference to FIGS. 3A-3F.

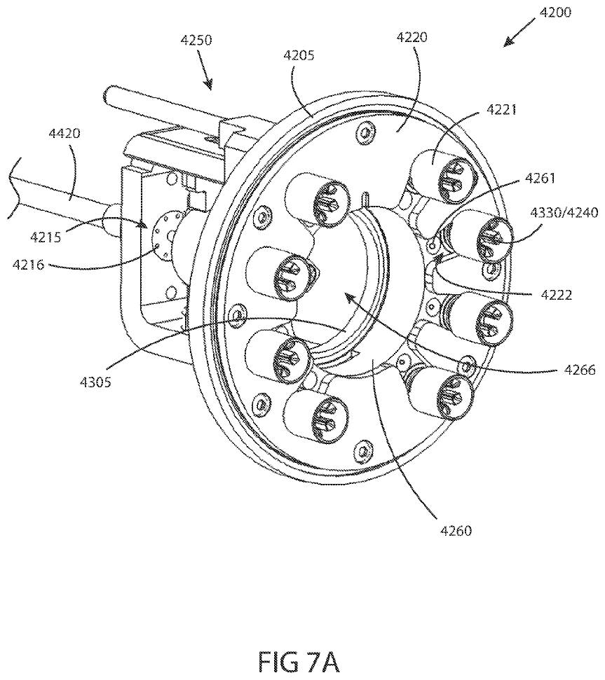

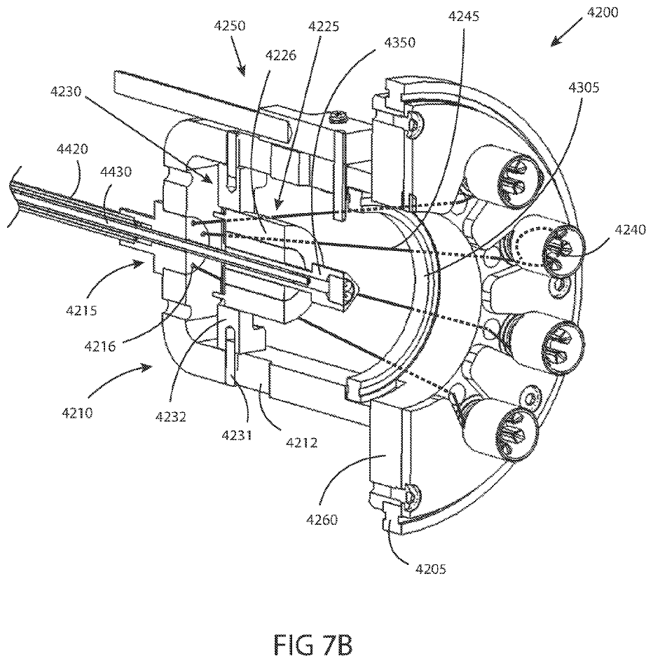

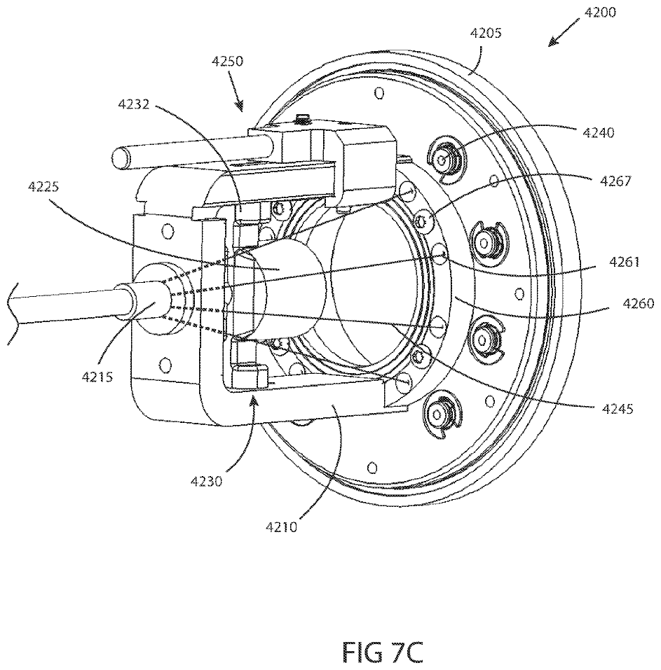

In some embodiments, the outer rotating assembly 4200 is constructed and arranged to operably engage the one or more control cables 445 of the tool 400 such as eight cables 4245, as described herebelow in reference to FIG. 7, the cables 4245 in turn being operably attached to the articulatable links 4510 of the tool 400. In some embodiments, the outer rotating assembly 4200 is constructed and arranged to rotate, relative to the inner rotating assembly 4300 and the interface assembly 4100, while maintaining the operative engagement of the cables 4245. In some embodiments, the rotation of the outer rotating assembly 4200 also rotates an outer shaft assembly 4420 and the articulatable links 4510. The components of the outer rotating assembly 4200 are described in detail herebelow in reference to FIGS. 7A-7C.

In some embodiments, a locking assembly 4250 comprises a handle 4251 fixedly attached to a slide 4252. The slide 4252 can be positioned on the support assembly 4210 of the outer rotating assembly 4200 and the pin extends through a slot in the support assembly. The handle 4251 may be slidingly positioned on a hub 4310 of inner rotating assembly 4300, and fixedly attached to a pin 4253, extending through a slot 4211 of the hub 4310. In some embodiments, the inner rotating assembly 4300 includes a capture port 4319 to slidingly receive the pin 4253. When the locking assembly 4250 is in a locked position (as shown), the pin 4253 engages the capture port 4319 and locks the orientation of the outer rotating assembly 4200 relative to the inner rotating assembly 4300 such that they rotate together, with respect to the interface assembly 4100. The locking assembly 4250 and the capture port 4319 are described in greater detail herein.

In some embodiments, the control assembly 410 operably attaches to a tool drive 200, such as described in applicant's co-pending application U.S. Provisional Application No. 62/614,228, filed Jan. 5, 2018, the content of which is incorporated herein by reference in its entirety. One or more gears of the tool drive 200 can operably engage one or more capstans 4330/4240 to control the rotation of each. In some embodiments, the tool drive 200 is constructed and arranged to rotate to in turn cause the rotation of the inner rotating assembly 4300 and/or the outer rotating assembly 4200.

In some embodiments, the shaft 440 comprises the outer shaft assembly 4420, and the inner shaft assembly 4430. In some embodiments, the support assembly 4210 comprises a motion compensation assembly 4230, constructed and arranged to compensate for changes in length between the outer shaft assembly 4420 and the inner shaft assembly 4430. For example, as the inner shaft assembly 4430 is steered and/or otherwise brought under tension, the motion compensation assembly 4230 may translate proximally to accommodate for a corresponding shortening of the outer shaft assembly 4420.

Referring to FIG. 3A, a perspective of view of an inner rotating assembly 4300 is illustrated, in accordance with embodiments of the present inventive concepts. In some embodiments, the inner rotating assembly 4300 comprises the hub 4310. The hub 4310 may be rotatably attached to the second bearing 4305. The hub 4310 may be operably attached to one or more capstans 4330 (four shown). One or more control cables 4345 (four in the embodiment shown, only one shown for illustrative clarity), exit the shaft 440 (extending proximally), extend through a central lumen 4311 of the hub 4310, extend further through lumen 4311, and each operably engage a capstan 4330.

In some embodiments, the inner rotating assembly 4300 can comprise a proximal cover 4320. In some embodiments, the proximal cover 4320 comprises one or more projections 4321, surrounding at least a portion of the capstans 4330. When attached, the proximal cover 4320 and the hub 4310 can define a space 4322, as shown in FIG. 3F, through which the one or more control cables 4345 can extend from the one or more capstans 4330 into the lumen 4311.

In some embodiments, the outer rotating assembly 4200 can comprise a slip ring 4225, configured to rotatably transfer electrical power and/or data to the inner rotating assembly 4300, such that the inner rotating assembly 4300 can rotate indefinitely without cable restriction.

Referring additionally to FIGS. 3B and 3C, front and back perspective views of a hub 4310 are illustrated, respectively, a proximal cover 4320 and capstans 4330 removed for illustrative clarity, in accordance with embodiments of the present inventive concepts . In some embodiments, the hub 4310 comprises a cylindrical structure with the central lumen 4311, and one or more ports 4315, positioned radially about the central lumen 4311. In some embodiments, the central lumen 4311 comprises a flared proximal end. In some embodiments, the central lumen 4311 extends through a projection 4312. In some embodiments, the central lumen 4311 comprises a keyed distal portion 4313. The hub 4310 can comprise the capture port 4319 and a projection with a recess for slidingly receiving a locking pin, as described hereabove in reference to FIG. 2B. In some embodiments, the hub 4310 comprises a bearing surface 4317, slidingly received by the second bearing 4305. In some embodiments, the hub 4310 further comprises one or more screw holes 4318 for securing one or more components to the hub 4310, such as the second bearing 4305, secured via screws 4316 (as shown in FIG. 3E).

Referring additionally to FIG. 3D, a cross-sectional view of a hub 4310 is illustrated, in accordance with embodiments of the present inventive concepts. In some embodiments, the keyed distal portion 4313 slidingly receives a connector 4350. In some embodiments, the connector 4350 comprises a mating keyed proximal end, such that rotational force can be transferred between the hub 4310 and the connector 4350. In some embodiments, the connector 4350 is fixedly attached to an inner shaft 4431 of the inner shaft assembly 4430, and to an inner rotating portion of slip ring 4225, such that rotational force is transferred between the connector 4350 and the inner shaft assembly 4430 and an inner ring 4227 of slip ring 4225. In this embodiment, the inner shaft assembly 4430, the connector 4350, and the inner ring 4227 do not rotate relative to each other. In some embodiments, the connector 4350 translates within the keyed distal portion 4313, such as to accommodate for length differences between the outer shaft assembly 4420 (not shown) and the inner shaft assembly 4430, as described herein.

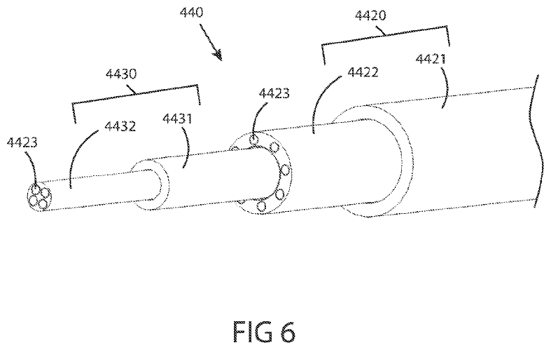

In some embodiments, the connector 4350 can comprise a radial adapter 4355, configured to guide cables 4345 from a radially compact configuration (e.g. aligned with one or more cable management lumens 4423 of an inner cable management shaft 4432, as described herebelow in reference to FIG. 6), to a radially dispersed position, oriented towards the one or more capstans 4330. In some embodiments, the lumen 4311 can comprise a flared (e.g. chamfered or filleted) proximal end to further guide the cables 4345 toward the one or more capstans 4330. The radial adapter 4355 is described in detail herebelow in reference to FIG. 5.

In some embodiments, the one or more cables 4345 extend proximally, from the end effector 460 (not shown in the present figure), through the inner shaft assembly 4430. The inner shaft assembly 4430 can comprise a first inner shaft 4431 and an inner cable management shaft 4432, as described herebelow in reference to FIG. 6. In some embodiments, the inner cable management shaft 4432 terminates distal to the radial adapter 4355, such as to allow for expansion, contraction, and/or to ease in manufacturing. In some embodiments, the cables 4345 extend proximally through the radial adapter 4355, out the proximal end of the lumen 4311, and each cable 4345 can operably engage (e.g. wrap around) a capstan 4330. In some embodiments, the one or more capstans 4330 can rotate to pull (tighten) and/or to feed (loosen) their corresponding cables 4345. Capstans 4330 can each be operably attached to a gear, such as a gear of the tool drive 200, as described herein.

In some embodiments, the hub 4310 can be constructed and arranged to energize (or otherwise provide power to) one or more cables 4345. For example, an end effector 460 can be energized with monopolar and/or bipolar energy to perform cautery or other energy delivery procedures. Conduit 4228, comprising one or more cables, can operably attach an outer ring 4226 of the slip ring 4225 to a source of monopolar, bipolar, or other energy. The inner ring 4227 can operably attach one or more cables 4341 to one or more brush assemblies 4340, each comprising a screw 4342.

Referring additionally to FIG. 3E, a perspective view of a hub 4310 is illustrated, in accordance with embodiments of the present inventive concepts. In some embodiments, the cable 4341 is electrically connected to the screw 4342, which is connected to a brush 4343. In some embodiments, the brush 4343 operably engages screw 4336E. The screw 4336E operably attaches to the cable 4345, as described in detail in reference to FIG. 3F, energizing the cable 4345 (when the cable 4345 is electrically conductive). The control cable 4345 transmits electrical energy to the end effector 460, as described herein. One or more components of the tool 400, such as the inner cable management shaft 4432, the radial adapter 4355, and/or the hub 4310 can comprise insulating material, and can be constructed and arranged to prevent two or more energized cables 4345 from touching (e.g. shorting the bipolar energy between them). Only one full brush assembly 4340 is shown for illustrative clarity. In some embodiments, there may be additional full brush assemblies 4340.

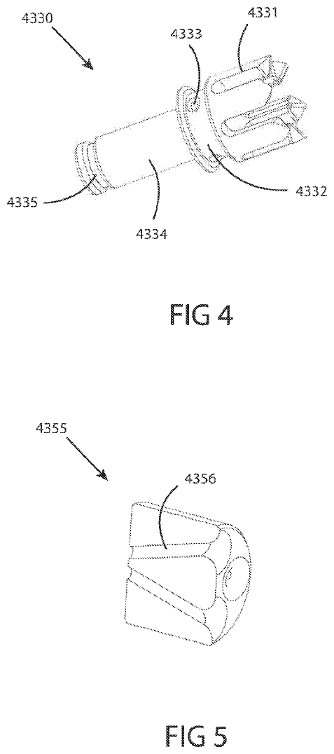

Referring additionally to FIG. 3F, a perspective cross-sectional view of a hub 4310 is illustrated, in accordance with embodiments of the present inventive concepts. In some embodiments, the one or more capstans 4330 are positioned within the one or more ports 4315. A detail view of the one or more capstans 4330 is shown in FIG. 4. The axle 4334 of each capstan 4330 is positioned within a corresponding port 4315, rotatably received therein, operably engaged by a third bearing 4339. In some embodiments, an o-ring 4338 surrounds axle 4334, and provides a rotation limiting (frictional) force between the one or more ports 4315 and the one or more capstans 4330. In some embodiments, a clip 4337 can operably engage the distal portion of the one or more capstans 4330 to maintain its position within the one or more ports 4315. A cable 4345 may be wound around each capstan 4330, and secured thereto via a screw 4336. In some embodiments, the cable 4345 can pass through the center of the one or more capstans 4330, and be secured beneath the screw 4336. As shown, in some embodiments, the screw 4336E comprises a longer screw (e.g. longer than the screw 4336), configured to extend distally beyond the one or more capstans 4330, and operably engage the brush assembly 4340 (as shown in FIG. 3E), electrifying the attached cable 4345.

Referring to FIG. 4, a perspective view of a capstan 4330 is illustrated, in accordance with embodiments of the present inventive concepts. In some embodiments, the one or more capstans 4330 comprise a cylindrical structure, comprising an axle 4334, extending distally from a winding surface 4332. One or more teeth, 4331 extend proximally from winding surface 4332, as shown. Axle 4334 can comprise a recess 4335 for engaging the clip 4337, as described hereabove in reference to FIG. 3F. In some embodiments, the winding surface 4332 can comprise one or more through holes 4333, for extending a cable 4345 therethrough, and securing to the one or more capstans 4330, such as via the screw 4336, as described hereabove in reference to FIG. 3F. Teeth 4331 can have rounded proximal tips, such as to ease mating with gears of the tool drive 200.

Referring to FIG. 5, a perspective cross-sectional view of a radial adapter 4355 is illustrated, in accordance with embodiments of the present inventive concepts. In some embodiments, the radial adapter 4355 can comprise one or more channels 4356 configured to slidingly receive and guide cables 4345 from a radially compact configuration to a radially dispersed configuration. In some embodiments, a distal end of the radial adapter 4355 can comprise one or more channels 4356 in a radially compact configuration that transition to a radially dispersed configuration at the proximal end of the radial adapter 4355. Radial adapter 4355 can comprise a low-friction material.

Referring to FIG. 6, a perspective partial cut away view of a portion of a shaft 440 is illustrated, in accordance with embodiments of the present inventive concepts. In some embodiments, the shaft 440 comprises the outer shaft assembly 4420, comprising a first shaft 4421 surrounding an outer cable management shaft 4422, comprising one or more cable management lumens 4423. In some embodiments, the shaft 440 further comprises the inner shaft assembly 4430, comprising the inner shaft 4431 surrounding the inner cable management shaft 4432, comprising one or more cable management lumens 4423. The first shaft 4421 and the outer cable management shaft 4422 can be fixedly attached to each other. The inner shaft 4431 and the inner cable management shaft 4432 can be fixedly attached to each other. In some embodiments, the inner shaft assembly 4430 is rotatably positioned within the outer cable management shaft 4422.

In some embodiments, the first shaft 4421 and the inner shaft 4431 each comprise a laser cut shaft, a torque wire type construction, and/or a polymer shaft. In some embodiments, the first shaft 4421 and the inner shaft 4431 can be lubricious for being slidingly received within a working channel as describe herein, and also transmit torque along its length. In some embodiments, shafts 4421, 4431 are flexible but still transmit torque.

Referring to FIG. 7A, a rear perspective view of an outer rotating assembly 4200 is illustrated, in accordance with embodiments of the present inventive concepts. In some embodiments, the outer rotating assembly 4200 comprises a second hub 4260. In some embodiments, the second hub 4260 comprises a ring-like structure, with a central opening 4266. In some embodiments, the inner rotating assembly 4300 can be rotatably positioned within the central opening 4266. In some embodiments, the tool 400 doesn't have the inner rotating assembly 4300, as well as one or more other components (such as the motion compensation assembly 4230 and/or the inner shaft assembly 4430), and the central opening 4266 provides access into the outer shaft assembly 4420.

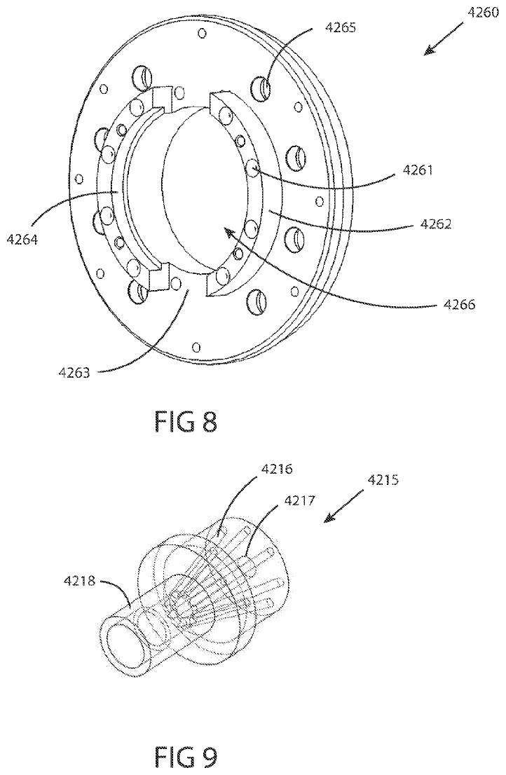

In some embodiments, the outer rotating assembly 4200 can comprise one or more capstans 4240, eight shown, in a pattern about the circumference of the outer rotating assembly 4200. The pattern can match a pattern of gears in the tool drive 200, as described herein and as described in applicant's co-pending application U.S. Provisional Application No. 62/614,228, filed Jan. 5, 2018, the content of which is incorporated herein by reference in its entirety. In some embodiments, the one or more capstans 4240 are of similar construction and arrangement to the capstans 4330, positioned within ports 4265 of the second hub 4260, as described herebelow in reference to FIG. 8. In some embodiments, the capstan assemblies include bearings, clips, o-rings, and screws for securing control cables 4245 thereto, similar to as described in reference to the capstans 4330 herein. In some embodiments, the outer rotating assembly 4200 operably engages one or more control cables 4245, such as eight cables 4245, to one or more links 4510 of the tool 400, as described herebelow in reference to FIG. 10.

In some embodiments, the outer rotating assembly 4200 comprises a proximal cover 4220, comprising one or more projections 4221, surrounding at least a portion of the one or more capstans 4240. The proximal cover 4220 can comprise one or more recesses 4222 through which the cables 4245 can extend from the capstans 4240, through holes 4261 in the second hub 4260, to a radial adapter 4215.

Referring to FIG. 7B, a perspective cross-sectional view of an outer rotating assembly 4200 is illustrated, in accordance with embodiments of the present inventive concepts. Referring additionally to FIG. 7C, a front perspective view of the outer rotating assembly 4200 is illustrated, in accordance with embodiments of the present inventive concepts. In some embodiments, the one or more control cables 4245 (eight in the embodiment shown, some removed for illustrative clarity), exit the outer shaft assembly 4420, and extend through the radial adapter 4215. The one or more cables 4245 may extend from the radial adapter 4215 to and through the holes 4261 of the second hub 4260. Each of the one or more cables 4245 can operably engage the one or more corresponding capstans 4240, as described herein.

In some embodiments, the support assembly 4210 supports a linear compensation mechanism 4230, comprising a yoke 4232, slidingly attached to the support assembly 4210. Pins 4231 extend from the yoke 4232 through slots 4212 in the support assembly 4210. The linear compensation mechanism 4230 operably attaches to the connector 4350, such that the linear shaft assembly 4430 can translate proximally and/or distally relative to the second hub 4260 and the outer shaft assembly 4420. For example, the distal ends of the outer shaft assembly 4420 and the inner shaft assembly 4430 can be fixed to each other, such that when the outer shaft assembly 4420 shortens under compression due to tensioning the cables 4245, the proximal end of the inner shaft assembly 4430 (including the connector 4350) moves proximally towards the second hub 4260.

Referring to FIG. 8, a perspective view of a second hub 4260 is illustrated, in accordance with embodiments of the present inventive concepts. In some embodiments, the second hub 4260 comprises a ring-like structure with the central opening 4266. A projection 4262 extends from the second hub 4260, defining a second bearing surface 4264. The second bearing 4305 is slidingly received by the second bearing surface 4264, and secured with one or more screws 4267 (as shown in FIG. 7C). The projection 4262 can comprise one or more recesses 4263, for example at positions where the support assembly 4210 operably attaches to the second hub 4260. The second hub 4260 comprises one or more ports 4265, as described herein.

Referring to FIG. 9, a partially transparent perspective view of a radial adapter 4215 is illustrated, in accordance with embodiments of the present inventive concepts. In some embodiments, the radial adapter 4215 comprises a cylindrical structure, with a central lumen 4217 and a neck 4218 extending distally. In some embodiments, the proximal end of the outer shaft assembly 4420 is fixedly attached within the neck 4218, and the inner shaft assembly 4430 extends from the outer shaft assembly 4420 and through the central lumen 4217, slidingly and rotatably positioned therein. In some embodiments, inner shaft assembly 4430 is free to rotate with respect to central lumen 4217, but is not free to translate with respect to central lumen 4217. In some embodiments, the radial adapter 4215 comprises one or more channels 4216, eight shown, one per cable 4245, and extends from a radially compact orientation to a radially dispersed orientation as shown.

Material for this component is low friction to reduce drag on cables 4245 and to enable the inner shaft assembly 4430 to rotate within this component.

Referring to FIG. 10, a perspective view of a distal portion of a tool 400 including an articulating portion 450 and an end effector 460 is illustrated, in accordance with embodiments of the present inventive concepts. Referring additionally to FIG. 10A, a perspective, partial sectional view of multiple articulatable links 4510 is illustrated, in accordance with embodiments of the present inventive concepts. Some of the cables 4245, 4345 have been removed for illustrative clarity. Referring additionally to FIG. 10B, a close-up perspective view of two neighboring articulatable links 4510 is illustrated, in accordance with embodiments of the present inventive concepts.

In some embodiments, the articulating section 450 can include a proximal articulating section 4501, and a distal articulating section 4502. The proximal articulating section 4501 is controlled by a first set of cables 4245a, terminating at the distal end of the proximal articulating section 4501, at a termination point 4246a. The distal articulating section 4502 is controlled by a second set of cables 4245b, terminating at the distal end of the distal articulating section 4502, at a termination point 4246b.

The proximal articulating section 4501 can comprise a proximal link 4505, configured to operably attach to the distal end of the first shaft 4421. In some embodiments, the cables 4245a,b can transition within the proximal link 4505 from an equally, radially spaced pattern within the outer cable management shaft 4422, as shown in FIG. 6, to the paired pattern, spaced every 90°, as shown in FIG. 10A. The proximal link 4505 can provide a hollow space and/or one or more channels for the transition of cables 4245a,b from equally spaced to paired. In some embodiments, the proximal articulating section 4501 can comprise more or less links than the distal articulating section 4502.

In some embodiments, the multiple articulatable links 4510 can each comprise a first articulating surface 4511, and a second articulating surface 4512. The first articulating surface 4511 and the second articulating surface 4512 can each comprise a convex profile (such as to facilitates a rolling interface to reduce friction between neighboring links). In some embodiments, the curve of the first articulating surface 4511 may be 90° offset from the curve of the second articulating surface 4512. The alternating links 4510 align as shown, with the first articulating surface 4511 and the second articulating surface 4512 mating to form an articulating joint. The multiple links 4510 allow articulation in two directions, with each alternating joint articulating 90° from its neighbor. In some embodiments, the first articulating surface 4511 and the second articulating surface 4512 are not offset 90°, such that a set of links only articulates in a single plane. In these embodiments, a section with non-alternating links 4510 can provide a tighter overall radius of curvature than the alternating links 4510. In some embodiments, the links 4510 of the proximal articulating section 4501 are not alternating, and the links 4510 of the distal articulating section 4502 are alternating. Each link 4510 can comprise one or more control cable channels 4513, and a central lumen 4514. The inner shaft assembly 4430 can be slidingly received within the central lumen 4514 throughout the proximal articulating section 4501 and the distal articulating section 4502.

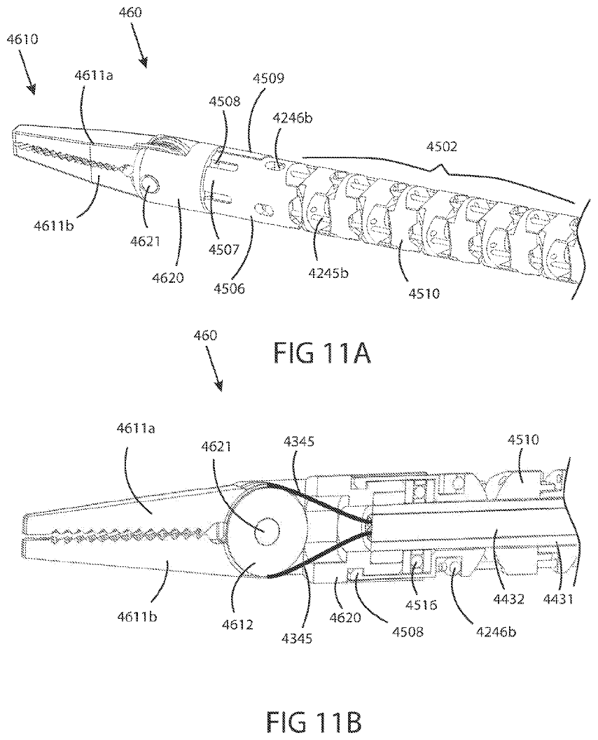

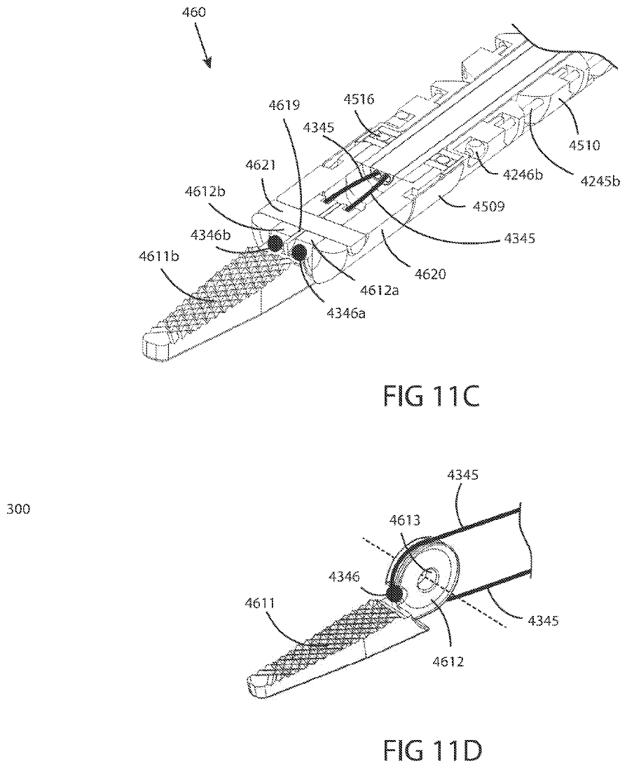

Referring to FIGS. 11A-D, perspective, sectional views of a distal portion of a tool 400, and perspective sectional views of an articulating jaw assembly 4610 and control cables 4345 are illustrated, in accordance with embodiments of the present inventive concepts.

In some embodiments, the end effector 460 comprises an articulating jaw assembly 4610, comprising jaws 4611a,b, each comprising a hub 4612, with a hole 4613. In some embodiments, the end effector 460 includes a clevis 4620, with a pin 4621, extending through holes 4613 of the jaws 4611a,b, defining an axis about which the jaws 4611a,b articulate. The jaws 4611a,b can each articulate approximately 180° about the pin 4621, in response to the control cables 4345. The control cables 4345 engage the hubs 4612 and secure to the distal side of the hubs 4612 at a fixation point 4246, such as the fixation point 4246b shown. In some embodiments, two cables 4345 comprise a single cable, wrapped about the hub 4612 (e.g. similar to a pully), and the control cable 4345 is secured to the hub 4612 at a fixation point 4346 to prevent the control cable 4345 from slipping about the hub 4612.

In some embodiments, the distal articulating section 4502 can comprise a distal link 4506, configured to secure to and rotatably receive the clevis 4620. In some embodiments, the distal link 4506 can comprise one or more fingers 4507, each comprising an inward projection 4508, configured to “Clip” into a recess in the clevis 4620 as shown. In some embodiments, the distal link 4506 comprises a retention cuff 4509, shown partially removed for illustrative clarity, surrounding the fingers 4507, preventing the clevis 4620 from disengaging the distal link 4506. In some embodiments, the clevis 4620 is fixedly attached to the inner shaft 4431, such that rotation of the inner shaft 4431 causes the rotation of the clevis 4620 and therefore the articulating jaw assembly 4610. In some embodiments, the distal link 4506 can include a thrust bearing 4516, between the proximal end of the clevis 4620 and the distal link 4506, preventing or at least limiting binding between the clevis 4620 and the distal link 4506.

In some embodiments, the jaws 4611a,b are separated by a washer 4619 along the pin 4621. In some embodiments, the washer 4619 and other components of the end effector 460 can comprise insulative materials, electrically isolating the first jaw 4611a from the second jaw 4611b. In some embodiments, washer 4619, clevis 4620, and pin 4621 are all non-conductive. Electrified cables 4345 can electrify the jaws 4611a, b, to allow for mono and/or bi-polar cautery and/or other energy delivery.

In some embodiments, tool 400 does not include an end effector 460 (e.g. tool 400 is provided without an end effector). For example, distal link 4506 can engage a variety of available accessories or end effectors such as a camera, cold knife, suction/irrigation, etc. Inward projections 4508 can engage mating features on the accessories and end effectors.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient, comprising:
an articulating probe assembly, comprising:
an inner probe comprising multiple articulating inner links;
an outer probe surrounding the inner probe and comprising multiple articulating outer links; and
at least two working channels that exit a distal portion of the probe assembly, and
at least one tool configured to translate through one of the at least two working channels, wherein the at least one tool is robotically controlled.

2.-11. (canceled)
